(19) United States
(12) Patent Application Publication
(10) Pub. No.: US 2025/0333378 A1
(43) Pub. Date: Oct. 30, 2025

(54) HYDROXYL-CONTAINING THIOL-ALKENE ETHERS AND PROCESS OF MAKING AND USING SAME

(71) Applicant: Government of the United States as represented by the Secretary of the Air Force, Wright-Patterson AFB, OH (US)

(72) Inventors: Alexander T. Lonnecker, Edwards AFB, CA (US); Kamran B. Ghiassi, Palmdale, CA (US); Jacob C. Marcischak, Tehachapi, CA (US); Van Michael Q. Saludo, Lancaster, CA (US)

(21) Appl. No.: 19/061,433

(22) Filed: Feb. 24, 2025

Related U.S. Application Data
(60) Provisional application No. 63/639,964, filed on Apr. 29, 2024.

(51) Int. Cl. C07C 319/14; C08F 2/50; C08F 116/14

(52) U.S. Cl. CPC C07C 319/14; C08F 2/50; C08F 116/14

(57) ABSTRACT

The present invention relates to hydroxyl-containing thiol-alkene ethers and process of making and using same. Such monomers minimize the drawbacks of current monomers as they are highly miscible, stable and yet can be converted within seconds to polymeric materials by the addition of a radical initiator, heating and/or exposure to UV light. Polymers that are made from the disclosed monomers may be used to produce elastomers, adhesives and as resins for processes such as casting and 3D printing that are used to make articles. Furthermore, such monomers do not exhibit the objectionable odor that is normally associated with thiol containing resin materials and such monomers have a low viscosity so a diluent is not when required when a resin containing the disclosed monomers is used as resin for casting and 3D printing. As a result, articles that are made from such resins do not undergo dimensional and/or structural changes that occur when diluents are removed during final processing.

### CROSS-REFERENCE TO RELATED APPLICATION

The present application claims priority to U.S. Provisional Application Ser. No. 63/639,964 filed Apr. 29, 2024, the contents of which is hereby incorporated by reference in their entry.

### RIGHTS OF THE GOVERNMENT

The invention described herein may be manufactured and used by or for the Government of the United States for all governmental purposes without the payment of any royalty.

### FIELD OF THE INVENTION

The present invention relates to hydroxyl-containing thiol-alkene ethers and process of making and using same.

### BACKGROUND OF THE INVENTION

Monomers are used to produce homopolymers and copolymers. Unfortunately, many current monomers are difficult to process as they are not as miscible as desired or they are solids. As a result, a new monomer that minimizes the drawbacks of current monomers was desired. Applicants recognized that in order to produce a stable monomer that had the desired characteristics, the monomer needed to be stabilized. Applicants recognized that the source of the stability problem was the excess reactivity of thiols with alkenes. Applicants solved this problem by adding a material that impeded the formation of thiol radicals. Applicants' monomers can be converted within seconds to polymeric materials by the addition of a radical initiator, heating and/or exposure to UV light.

Polymers that are made from Applicants' monomers may be used to produce elastomers, adhesives and as resins for processes such as casting and 3D printing that are used to make articles. For example, 3-dimensional (3D) printing makes three-dimensional objects by building up material, based upon design data provided from a computer aided design (CAD) system. One technique is to deposit a crosslinkable material in a predetermined pattern, according to design data provided from a CAD system, with the build-up of multiple layers forming the object. The crosslinkable materials can be either in the form of filaments, powdered resins or liquid resins. When liquid resins are used as a raw material, the additive manufacturing processes that are used include direct ink writing and vat photopolymerization (VP)—examples of VP include but are not limited to techniques such as stereolithography (SLA), digital light processing (DLP), masked stereolithogrpahy (MSLA), and liquid crystal display (LCD) 3D printing.

Unlike other resin materials, Applicants' resin monomer has a low viscosity. As a result, a diluent is not when required when Applicants' resin is used as resin for casting and 3D printing. Thus, articles that are made from Applicants' resins do not undergo dimensional and/or structural changes that occur when diluents are removed during final processing. Furthermore, most thiol containing resin materials have a negative reactivity and an objectionable odor. The higher molecular weight of Applicants' resin material minimizes both of such drawbacks.

### SUMMARY OF THE INVENTION

The present invention relates to hydroxyl-containing thiol-alkene ethers and process of making and using same. Such monomers minimize the drawbacks of current monomers as they are highly miscible, stable and yet can be converted within seconds to polymeric materials by the addition of a radical initiator, heating and/or exposure to UV light. Polymers that are made from the disclosed monomers may be used to produce elastomers, adhesives and as resins for processes such as casting and 3D printing that are used to make articles. Furthermore, such monomers do not exhibit the objectionable odor that is normally associated with thiol containing resin materials and such monomers have a low viscosity so a diluent is not when required when a resin containing the disclosed monomers is used as resin for casting and 3D printing. As a result, articles that are made from such resins do not undergo dimensional and/or structural changes that occur when diluents are removed during final processing.

### BRIEF DESCRIPTION OF THE DRAWINGS

The accompanying drawings, which are incorporated in and constitute a part of this specification, illustrate embodiments of the invention and, together with the summary given above, and the detailed description given below, serve to explain the invention.

FIG. 1 is a process diagram depicting a process for producing a resin that can be cured into an article.

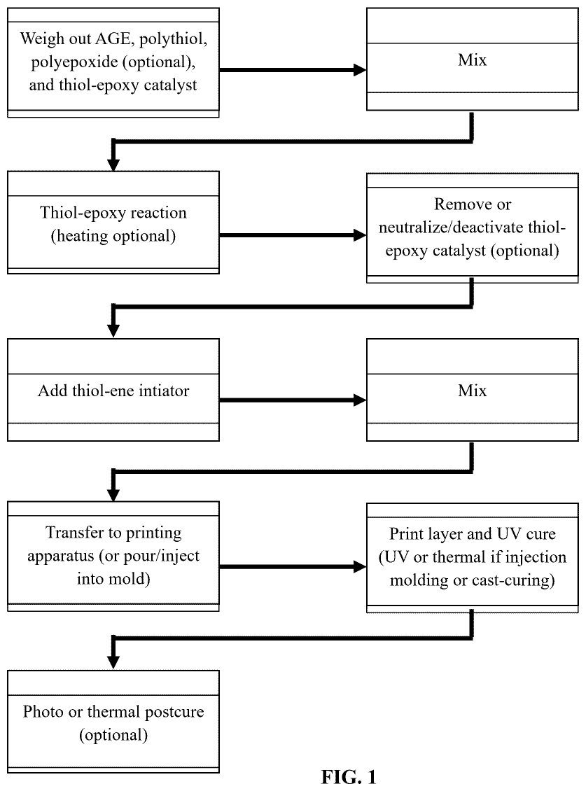

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

### Definitions

Unless specifically stated otherwise, as used herein, the terms “a”, “an” and “the” mean “at least one”.

As used herein, the terms “include”, “includes” and “including” are meant to be non-limiting.

As used herein, the words “about,” “approximately,” or the like, when accompanying a numerical value, are to be construed as indicating a deviation as would be appreciated by one of ordinary skill in the art to operate satisfactorily for an intended purpose.

As used herein, the words “and/or” means, when referring to embodiments (for example an embodiment having elements A and/or B) that the embodiment may have element A alone, element B alone, or elements A and B taken together.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Monomer, Polymer, Articles and Resin

For purposes of this specification, headings are not considered paragraphs. In this paragraph Applicants disclose a monomer having the following structure:

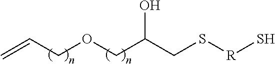

wherein each indice n is independently an integer from 1 to 4, preferably each indice n is 1; R is C₂₋₁₀ n-alkanediyl, C₃₋₁₀ branched alkanediyl, C₆₋₁₀ cyclalkanediyl, C₆₋₁₀ alkanecycloalkanediyl, or [(CH₂—)ₚ—X—]_{q}—(—CH₂—)—, wherein each X is independently:

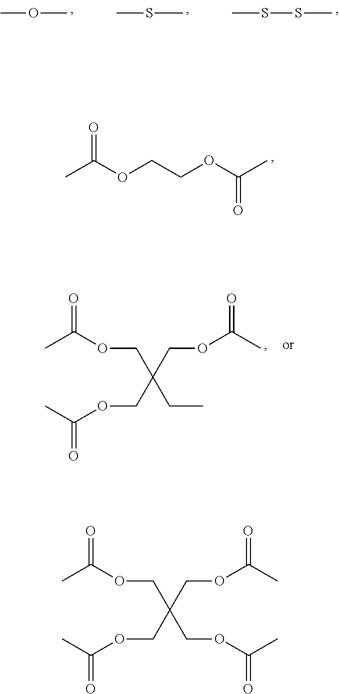

q is an integer from 1 to 5, preferably q is 1; each p is independently an integer from 2 to 6, preferably each p is independently an integer from 2 to 3, more preferably each p is 2.

In this paragraph, Applicants disclose a polymer comprising a monomer according to the previous paragraph. This polymer can be a homopolymer or copolymer.

In this paragraph, Applicants disclose a polymer according to the previous paragraph, said polymer being an epoxide functionalized polymer comprising a backbone and chain ends, said backbone and/or said chain ends comprising one or more epoxide groups, in one aspect, said epoxide functionalized polymer comprises side chains, said epoxide functionalized polymer comprising side chains, comprising one or more epoxide groups on said side chains, backbone and/or said chain ends, in one aspect, said epoxide functionalized polymer is selected from the group consisting of a polysilane, a polyether, a polymethacrylates and mixtures thereof, preferably said epoxide functionalized polymer is selected from the group consisting of a polysilane, a polyether, and mixtures thereof. The polysilanes and polyethers can, due to monomer/polymer miscibility, can provide improved thermomechanical properties.

In this paragraph, Applicants disclose an article comprising a polymer according to the previous two paragraphs, preferably said article is selected from a seal, a gasket, a hose, a grip, a bushing, a bumper, a tire, an electronic matrix, and/or a bladder.

In this paragraph, Applicants disclose a finished article comprising a polymer according to paragraphs of this section titled “Monomer, Polymer, Articles and Resin” that disclosing a polymer and/or an article according to the previous paragraph, preferably said finished article is an aerospace vehicle or a computer system, preferably said aerospace vehicle is a manned, or unmanned, fixed-wing aircraft; a manned, or unmanned, rotary wing aircraft (i.e., helicopters, quadcopters, etc.); combination fixed-wing aircraft and rotary-wing aircraft, manned or unmanned (i.e., compound helicopters, tiltrotor aircraft, etc.); subsonic missile, supersonic missile, or hypersonic missile; solid fueled rocket, liquid fueled rocket, or gas fueled rocket, such rockets maybe manned or unmanned; or a lighter than air aerospace vehicle, (i.e., blimps, such lighter than air aerospace vehicle maybe manned or unmanned).

In this paragraph, Applicants disclose a resin comprising a monomer according to the first paragraph of this section titled “Monomer, Polymer, Articles and Resin”, a radical scavenger, preferably said radical scavenger is selected from the group consisting of phenothiazine, quinone monomethyl ether, butylated hydroxytoluene, hydroquinone, oil red 40, acid yellow 24, and mixtures thereof, most preferably said radical scavenger is selected from the group consisting of acid yellow 24, said radical scavenger being preferably present in a concentration, based on total resin weight, of about 0.001% to about 0.1%; a base, preferably said base is tertiary amine and/or a uracyl base catalyst, more preferably said base is N,N-dimethylethanolamine (DMEA), triethylene diamine (TEDA), bis(2-dimethylaminoethyl)ether (BDMEE), N-ethylmorpholine, N′,N′-dimethylpiperazine, N,N,N′,N′,N′-pentamethyl-diethylene-triamine (PMD ETA), N,N-dimethylcyclohexylamine (DMCHA), N,N-dimethylbenzylamine (DMBA), N,N-dimethylcethylamine, N,N,N′,N″,N″-pentamethyl-dipropylene-triamine (PMDPTA), triethylamine, 1-(2-hydroxypropyl)imidazole, 1,8-diazabicyclo[5.4.0]undec-7-ene bicarbonate (DBU), 1,4-diazabicyclo[2.2.2]octane, more preferably said base is a tertiary amine catalyst, most preferably said base is triethylamine; said base being preferably present in a concentration, based on total resin weight, of about 0.1% to about 10%, preferably when said base has a pKa greater than 30, said base is present in a concentration, based on total resin weight, of about 0.1% to about 1%, preferably when said base has a pKa of 30 or less, said base is present in a concentration, based on total resin weight, of greater than 1% to about 5%. The base and/or radical scavenger stabilizes said monomer. Thus, allowing said monomer to be stored prior to use.

In this paragraph, Applicants disclose a resin according to the previous paragraph, said resin comprising a filler, preferably said filler is selected from the group consisting of carbon black, calcium carbonate, silica, fumed silica, mica, polymer powders, molecular sieve powders, titanium dioxide, alkaline blacks, cellulose, zinc sulfide, heavy spar, alkaline earth oxides and mixtures thereof, preferably said resin comprises, based on total resin weight, from about 1% to about 70% filler, more preferably said resin comprises, based on total resin weight, from about 10% to about 50% filler.

### Process of Making

In this paragraph, Applicants disclose a process of making a polymer comprising neutralizing and/or removing the base and/or radical scavenger of the resin disclosed in the paragraphs of this application within the section titled “Monomer, Polymer, Articles and Resin”; optionally, adding a thermal initiator and/or photo initiator to said resin, preferably said thermal initiator is selected from the group consisting of 2,2′-Azobis(2-methylpropionitrile), benzoyl peroxide and mixtures thereof; and preferably said photo initiator is selected from the group consisting of 2,2-Dimethoxy-2-phenylacetophenone, Diphenyl(2,4,6-trimethylbenzoyl)phosphine oxide and mixtures thereof, preferably, based on total resin weight, said resin comprises from about 0.01% to about 1% of said optional thermal initiator and/or photo initiator, more preferably based on total resin weight, said resin comprises from about 0.1% to about 1% of said optional thermal initiator and/or photo initiator; and exposing said resin to UV light; and/or heating said resin.

In this paragraph, Applicants disclose a process of making the monomer disclosed the paragraph of the section of this application titled “Monomer, Polymer, Articles and Resin”, said process comprising combining a monomer comprising allyl moieties and epoxide moieties, said monomer having the following formula:

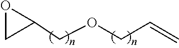

wherein each indice n is independently an integer from 1 to 4, preferably each indice n is 1; a polythiol comprising thiol moieties, said polythiol having the following formula:

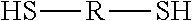

wherein R is C₂₋₁₀ n-alkanediyl, C₃₋₁₀ branched alkanediyl, C₆₋₁₀ cyclalkanediyl, C₆₋₁₀ alkanecycloalkanediyl, or [(CH₂—)ₚ—X—]_{q}—(—CH₂—)—, wherein each X is independently:

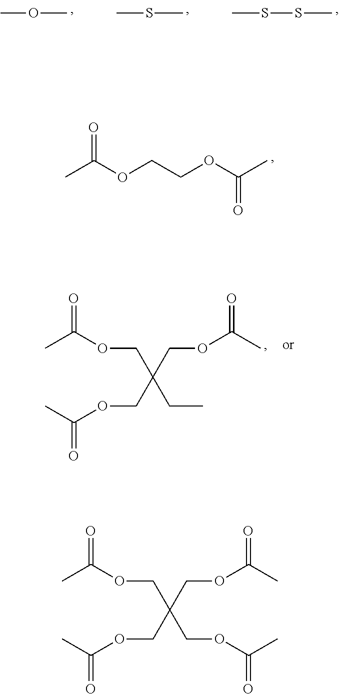

q is an integer from 1 to 5, preferably q is 1; each p is independently an integer from 2 to 6, preferably each p is independently an integer from 2 to 3, more preferably each p is 2; a base and/or radical scavenger; and an optional polyepoxide; wherein the ratio of the sum of said monomer's allyl moieties and epoxide moieties and said epoxide moieties of said polyepoxide, to said thiol moieties of said polythiol being from about 0.8:1 to about 1.2:1, preferably the ratio of the sum of said monomer's allyl moieties and epoxide moieties and said epoxide moieties of said polyepoxide, to said thiol moieties of said polythiol being from about 1:1 to about 0.9:1; said base and/or radical scavenger being present at a level of about 0.1 mole percent to about 1 mole percent based on total mole percent of said monomer comprising allyl moieties and epoxide moieties, preferably said base and/or radical scavenger being present at a level of about 0.1 mole percent to about 0.5 mole percent based on total mole percent of said monomer comprising allyl moieties and epoxide moieties. Said process may comprise heating said combined monomer comprising allyl moieties and epoxide moieties, said polythiol comprising thiol moieties, said base and/or radical scavenger and said optional polyepoxide to a temperature of about −10° C. to about 60° C. for a time of from about 5 minutes to about 72 hours, preferably said process may comprise heating said combined monomer comprising allyl moieties and epoxide moieties, said polythiol comprising thiol moieties, said base and/or radical scavenger and said optional polyepoxide to a temperature of about 50° C. for a time of from about 1 to about 2 hours.

In this paragraph, Applicants disclose the process of the previous paragraph wherein said monomer comprising allyl moieties and epoxide moieties is an allyl glycidyl ether.

In this paragraph, Applicants disclose a process of making an article comprising casting, 3D printing, injection molding, spray coating the resin disclosed in the paragraphs of this application within the section titled “Monomer, Polymer, Articles and Resin”.

### Test Method to Determine Reaction Completion

For purposes of the present application, epoxide and allyl group content of a liquid resin is determined as follows: Nuclear magnetic resonance spectroscopy will be taken during the preparation of the resin and used to monitor the reaction of the thiol and epoxide functional groups of allyl glycidyl ether. The peaks corresponding to the alkene protons in allyl glycidyl ether can be found at 5.9 ppm (1H, m, CH₂═CH) and 5.3-5.15 ppm (2H, m, CH₂═CH). Set the integration of the alkene protons to values of one and two for the signals at 5.9 ppm and 5.3-5.14 ppm, respectively. The peak corresponding to the protons in the epoxide ring of allyl glycidyl ether [3.15 ppm (1H, m), 2.8 ppm (1H, dd), 2.65 ppm (1H, dd)] and adjacent to the ring [3.7 ppm (1H, dd, CH—CH₂—O), 3.4 ppm (1H, dd, CH—CH₂—O)] will be integrated and used to monitor the thiol-epoxide ring opening reaction. At the beginning of the reaction, before addition of the thiol, integration values of each of the epoxide signals will be one. Complete ring-opening of allyl glycidyl ether can be determined when the three proton signals of the glycidyl ether (3.15, 2.8, and 2.65 ppm) have an integration value of zero. Emergence of the methine proton signal [3.91 ppm (1H, m, —CH—OH)] and methylene protons signal [3.53-3.44 ppm (2H, m, —O—CH₂—CH—OH)] of the ring-opened product will coincide with the consumption of glycidyl ether protons and will integrate to a value of one and two at full conversion, respectively. The resin at this point is ready for thiol-ene polymerization and crosslinking.

For purposes of this application, the crosslink density is determined using the method outlined by Mujtaba et al., where the plateau modulus is determined using dynamic mechanical analysis, and the modulus is plugged in to the equation G vkT, where G is the rubbery plateau modulus, v is the crosslink density, k is the gas constant, and T is the temperature.

### EXAMPLES

The following examples and methods are presented as illustrative of the present invention or methods of carrying out the invention, and are not restrictive or limiting of the scope of the invention in any manner.

### Example 1. Reaction with Triethylamine (High Amount)

To a 250 mL round bottom flask, 47.449 g of ethylene glycol bis(3-mercaptopropionate), 4.873 g of pentaerythritol tetra(3-mercaptopropionate), and 2.361 g of triethylamine were added. 25.066 g of allyl glycidyl ether was then added, sealed and heated to 50° C. The reaction was stirred at temperature for 2 hours, or until complete conversion of the allyl glycidyl ether, at which point the reaction flask was allowed to cool to room temperature and the triethylamine was removed in vacuo. Diphenyl(2,4,6-trimethylbenzoyl)phosphine oxide (0.726 g, 1 wt %) was added and stirred until dissolved. The mixture was casted into molds and exposed to 405 nm light to polymerize. Fully cured samples were removed after a few minutes.

### Example 2—Reaction with Triethylamine (Low Amount)

To a 100 mL round bottom flask, 4.2637 g of 3,6-dioxa-1,8-octane-dithiol, 11.4284 g of pentaerythritol tetra(3-mercaptopropionate), and 0.0714 g of triethylamine were added. 7.9964 g of allyl glycidyl ether was then added and sealed. The reaction was stirred at room temperature for 72 hours, or until complete conversion of the allyl glycidyl ether, at which point triethylamine was removed in vacuo. Diphenyl(2,4,6-trimethylbenzoyl)phosphine oxide (0.726 g, 1 wt %) was added and stirred until dissolved. The mixture was casted into molds and exposed to 405 nm light to polymerize. Fully cured samples were removed after a few minutes.

### Example 3—Reaction with K₂CO₃

To a 250 mL round bottom flask, 34.880 g of 3,6-dioxa-1,8-octane-dithiol, 7.590 g of trimethylolpropane tris(3-mercaptopropionate), and 0.1538 g of potassium carbonate were added. 24.970 g of allyl glycidyl ether was then added, sealed, and heated to 50° C. The reaction was stirred at temperature for 2 hours, or until complete conversion of the allyl glycidyl ether, at which point the reaction flask was allowed to cool to room temperature and the potassium carbonate was removed by filtration. 2,2-Dimethoxy-2-phenylacetophenone (0.3545 g, 0.05 wt %) was added and stirred until dissolved. The mixture was casted into molds and exposed to 365 nm light to polymerize. Fully cured samples were removed after a few minutes.

### Example 4—Reaction with Amberlyst Resin

To a 250 mL round bottom flask, 28.5240 g of 3,6-dioxa-1,8-octane-dithiol, 15.2801 g of pentaerythritol tetra(3-mercaptopropionate), and 4.9171 g of Amberlyst IRA67 resin were added. 25.0171 g of allyl glycidyl ether was then added, sealed, and heated to 50 C. The reaction was stirred at temperature for 2 hours, or until complete conversion of the allyl glycidyl ether. The Amberlyst resin was removed by filtration and washed with acetone three times (3×50 mL). The resin was concentrated in vacuo to yield 56.4237 g of clear oil. Diphenyl(2,4,6-trimethylbenzoyl)phosphine oxide (0.6871 g, 1 wt %) was added and stirred until dissolved. The mixture was casted into molds and exposed to 405 nm light to polymerize. Fully cured samples were removed after a few minutes.

### Example 5—Reaction with BADGE

To a 20 mL scintillation vial, 2.958 g of bisphenol A diglycidyl ether, 1.850 g of 3,6-dioxa-1,8-octane-dithiol, 1.077 g of pentaerythritol tetra(3-mercaptopropionate), and 0.133 g of triethylamine were added. 0.526 g of allyl glycidyl ether was then added, sealed, and heated to 50° C. The reaction was stirred at temperature for 2.5 hours, or until complete conversion of the allyl glycidyl ether at which point the reaction flask was allowed to cool to room temperature and the triethylamine was removed in vacuo. Diphenyl(2,4,6-trimethylbenzoyl)phosphine oxide (0.0652 g, 1 wt %) was dissolved in a small amount of chloroform (0.5 mL), added to the resin, and mixed until dissolved. The acetone was removed by vacuum and the resin was injected into a mold. Exposure to 405 nm light led to the polymerization of the resin and fully cured samples were removed after a few minutes.

### Example 6—Reaction with Urethane

To a 20 mL scintillation vial, 0.6478 of hexamethylene diglycidyl urethane 2.6259 g, and 0.0189 g of potassium carbonate were added. The reaction was heated and stirred until the glycidyl urethane dissolved in the thiol. The glycidyl urethane fully reacted with the thiol, forming a slightly viscous suspension, after stirring at room temperature overnight. To this mixture, 1.8734 g of allyl glycidyl ether and 1.0045 g of pentaerythritol tetra(3-mercaptopropionate were added. After stirring for 2 hours at room temperature, the allyl glycidyl ether was consumed and the resin was filtered, yielding 5.8698 g of clear oil. Diphenyl(2,4,6-trimethylbenzoyl)phosphine oxide (0.0595 g, 1 wt %) was added and stirred until dissolved. The mixture was casted into molds and exposed to 405 nm light to polymerize. Fully cured samples were removed after a few minutes.

While the present invention has been illustrated by the description of one or more embodiments thereof, and while the embodiments have been described in considerable detail, they are not intended to restrict or in any way limit the scope of the appended claim to such detail. Additional advantages and modification will be readily apparent to those skilled in the art. The invention in its broader aspects is therefore not limited to the specific details, representative apparatus and methods and illustrative examples shown and described. Accordingly, departures may be made from such details without departing from the scope or the spirit of the general inventive concept exemplified herein.

What is claimed is:

1. A monomer having the following structure: 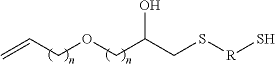
a) wherein each indice n is independently an integer from 1 to 4;
b) R is C₂₋₁₀ n-alkanediyl, C₃₋₁₀ branched alkanediyl, C₆₋₁₀ cyclalkanediyl, C₆₋₁₀ alkanecycloalkanediyl, or [(CH₂—)ₚ—X—]_{q}—(—CH₂—)—, wherein each X is independently 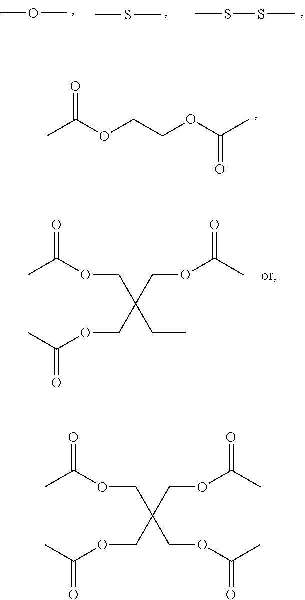
q is an integer from 1 to 5; and
each p is independently an integer from 2 to 6.

2. A polymer comprising a monomer according to claim 1.

3. The polymer of claim 2, said polymer being an epoxide functionalized polymer comprising a backbone and chain ends, said backbone and/or said chain ends comprising one or more epoxide groups, said epoxide functionalized polymer being selected from the group consisting of a polysilane, a polyether, a polymethacrylates and mixtures thereof.

4. A process of making a monomer according to claim 1, said process comprising combining:
a) a monomer comprising allyl moieties and epoxide moieties, said monomer having the following formula: 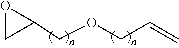
wherein each indice n is independently an integer from 1 to 4;
b) a polythiol comprising thiol moieties, said polythiol having the following formula 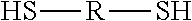
wherein R is C₂₋₁₀ n-alkanediyl, C₃₋₁₀ branched alkanediyl, C₆₋₁₀ cyclalkanediyl, C₆₋₁₀ alkanecycloalkanediyl, or [(CH₂—)ₚ—X—]_{q}—(—CH₂—)—, wherein each X is independently: 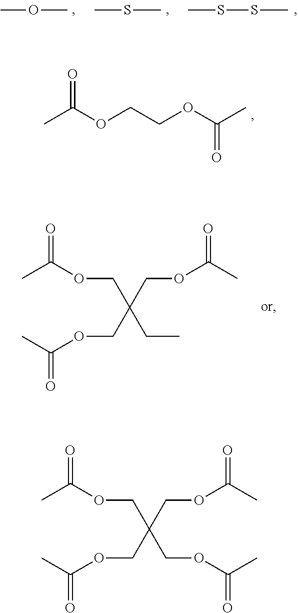
q is an integer from 1 to 5;
each p is independently an integer from 2 to 6;
c) a base and/or radical scavenger; and
d) an optional polyepoxide;
wherein the ratio of the sum of said monomer's allyl moieties and epoxide moieties and said epoxide moieties of said polyepoxide, to said thiol moieties of said polythiol being from about 0.8:1 to about 1.2:1; said base and/or radical scavenger being present at a level of about 0.1 mole percent to about 1 mole percent based on total mole percent of said monomer comprising allyl moieties and epoxide moieties.

5. The process of claim 4 wherein said monomer comprising allyl moieties and epoxide moieties is an allyl glycidyl ether.
